(19) United States
(12) Patent Application Publication
(10) Pub. No.: US 2025/0099774 A1
(43) Pub. Date: Mar. 27, 2025

(54) DEFIBRILLATOR AND DEFIBRILLATOR MANAGEMENT SYSTEM

(71) Applicant: NIHON KOHDEN CORPORATION, Tokyo (JP)

(72) Inventors: Kanchi ITO, Tokorozawa-shi, Saitama (JP); Yuji IGAWA, Tokorozawa-shi, Saitama (JP); Fumihito IWAI, Tokorozawa-shi, Saitama (JP)

(73) Assignee: NIHON KOHDEN CORPORATION, Tokyo (JP)

(21) Appl. No.: 18/833,736

(22) PCT Filed: Jan. 10, 2023
(86) PCT No.: PCT/JP2023/001771

§ 371 Date: Jul. 26, 2024

(30) Foreign Application Priority Data
Jan. 28, 2022 (JP) 2022-011702

(51) Int. Cl. A61N 1/39

(52) U.S. Cl. CPC A61N 1/3904; A61N 1/3925

(57) ABSTRACT

A defibrillator configured to execute a defibrillation process of delivering an electric shock for defibrillation to a heart of a subject. The defibrillator includes: a processor, and a memory that stores a computer-readable instruction that when executed by the processor, causes the defibrillator to perform operations including: receiving a raw data related to at least one of a plurality of components configuring the defibrillator and an accessory of the defibrillator; and transmitting the received raw data and an identification information of the defibrillator to a management server. The raw data is used to determine presence or absence of an abnormality in at least one of the plurality of components and the accessory.

### TECHNICAL FIELD The present disclosure relates to a defibrillator and a defibrillator management system.

### BACKGROUND ART

Currently, automated external defibrillators (hereinafter referred to as “AEDs”) have been rapidly widespread. Such an AED delivers a strong electric shock for defibrillation to the heart of a patient who has suffered a sudden cardiac arrest due to ventricular fibrillation, so as to restore a cardiac function of the patient. In addition, malfunction etc. of the defibrillator directly endangers the life of the patient who has suffered the cardiac arrest. Therefore, there have been known a system including an AED and a management server that periodically manages a state of the AED. For example, Japanese Patent No. 5432767 (Patent Document 1) discloses a remote maintenance system including an AED and a maintenance center communicatively connected to the AED.

In the remote maintenance system disclosed in the Patent Document 1, information indicating presence or absence of an abnormality in the AED is transmitted from the AED to the maintenance center via a relay unit, and the maintenance center sends an e-mail to the administrator of the AED when the information indicates an abnormality in the AED. In this manner, the AED administrator can grasp the abnormality of the AED by the email sent from the maintenance center.

However, while the above-described maintenance center can detect whether there is an abnormality in the AED based on the information transmitted from the AED, the maintenance center cannot handle applied data analysis such as abnormality prediction using a raw data related to components configuring the AED. From this point of view; there is room for further improvement of the defibrillator such as the AED and defibrillator management systems.

### SUMMARY

An object of the present disclosure is to provide a defibrillator and a defibrillator management system which can effectively use raw data related to components, etc. configuring the defibrillator such as the AED.

According to an aspect of the present disclosure, there is provided a defibrillator configured to execute a defibrillation process of delivering an electric shock for defibrillation to a heart of a subject. The defibrillator includes: a processor; and a memory that stores a computer-readable instruction that when executed by the processor, causes the defibrillator to perform operations including; receiving a raw data related to at least one of a plurality of components configuring the defibrillator and an accessory of the defibrillator; and transmitting the received raw data and an identification information of the defibrillator to a management server. The raw data is used to determine presence or absence of an abnormality in at least one of the plurality of components and the accessory.

According to the aforementioned configuration, the raw data related to at least one of the plurality of components configuring the defibrillator and the accessory of the defibrillator is transmitted to the management server. In this manner, data analysis such as abnormality prediction in the defibrillator can be performed in the management server by effectively using the raw data transmitted from the defibrillator. In this view point, in a conventional remote maintenance system including a defibrillator and a management server, applied data analysis such as abnormality prediction in the defibrillator cannot be performed, although the management server can detect abnormality of the defibrillator based on information indicating presence or absence of abnormality in the defibrillator. On the other hand, in the defibrillator according to this configuration, since it is possible to transmit the raw data related to at least one of the plurality of components and accessory to the management server, the management server can perform the data analysis such as abnormality prediction in the defibrillator by effectively using the raw data transmitted from the defibrillator. In this manner, it is possible to provide a defibrillator that can effectively use the raw data generated from components, etc. of the defibrillator.

According to an aspect of the present disclosure, there is provided a defibrillator management system including: the aforementioned defibrillator; and a management server communicatively connected to the defibrillator.

According to the aforementioned configuration, since it is possible to transmit the raw data related to at least one of the plurality of components and the accessory to the management server, the management server can perform data analysis such as abnormality prediction in the defibrillator by effectively using the raw data transmitted from the defibrillator. In this manner, it is possible to provide a defibrillator management system that can effectively use the raw data generated from the defibrillator, etc.

### BRIEF DESCRIPTION OF DRAWINGS

FIG. 1 is an example of a defibrillator management system according to one embodiment of the present disclosure.

FIG. 2 is a block diagram illustrating the configuration of the AED.

FIG. 3 is a flow chart for describing processes of a self-test executed by the AED.

FIG. 4 is an example of a defibrillator management table data.

### DESCRIPTION OF EMBODIMENT

An embodiment will be described below with reference the drawings. For convenience of explanation, dimensions of each member shown in each of the drawings may be different from actual dimensions of the member.

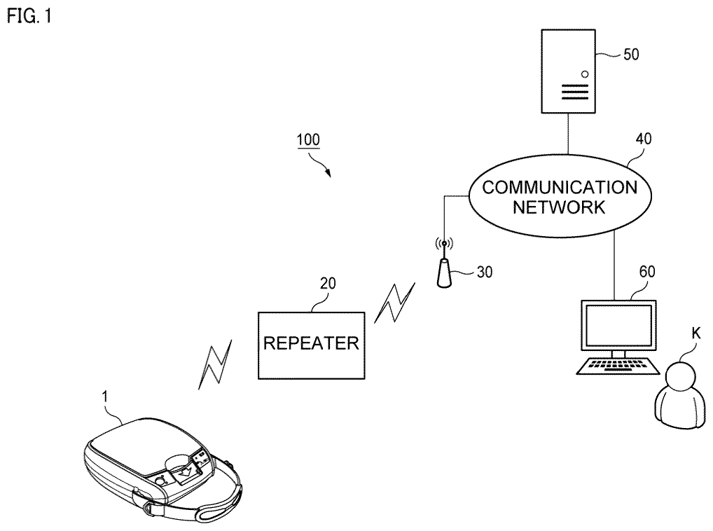

First, the configuration of a defibrillator management system 100 according to one embodiment of the present disclosure (hereinafter referred to as present embodiment) will be described below with reference to FIG. 1. FIG. 1 is an example of a defibrillator management system 100 according to present embodiment. In the defibrillation management system 100, an automated external defibrillator 1 (hereinafter referred to as AED 1) is communicatively connected to a repeater 20 through wireless communication, as shown in FIG. 1. The wireless communication between AED 1 and the repeater 20 may be a short-range wireless communication such as Bluetooth (registered trademark) and Wi-Fi (registered trademark).

The repeater 20 is communicatively connected to a wireless base station 30. The repeater 20 includes a wireless communication module for performing short-range wireless communication with the AED 1 arranged around the repeater 20, and a wireless communication module for performing wireless communication with the wireless base station 30 by an X-generation mobile communication system such as LTE and 5G. The repeater 20 is communicatively connected to a management server 50 arranged on the communication network 40 via the wireless base station 30. The communication network 40 includes a core network, the Internet, and the like.

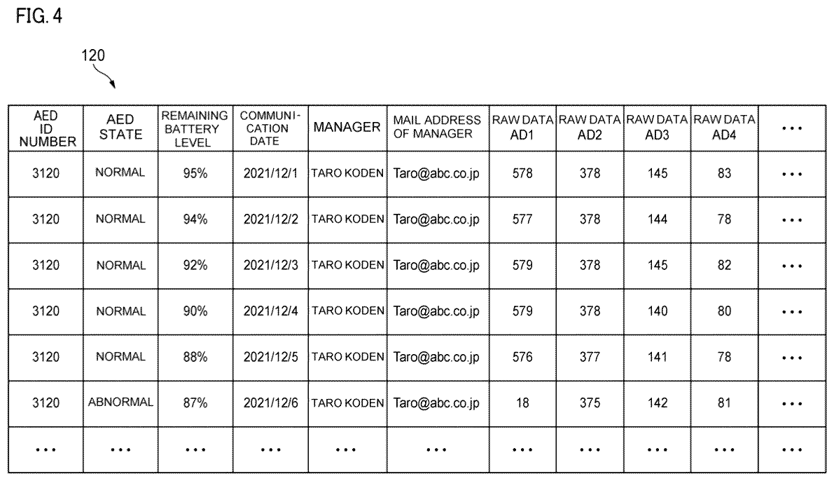

The management server 50 is a server that periodically manages a state of the AED 1 and is configured to receive information related to the AED 1 from the AED 1 via the repeater 20. The management server 50 is configured to store defibrillation management table data 120 (refer to FIG. 4), which will be described later. In addition, the management server 50 is configured to automatically perform applied data analysis such as abnormality prediction in the AED 1 based on the raw data related to the components transmitted from the AED 1.

The management server 50 is communicatively connected to an administrator terminal 60 via the communication network 40. The administrator terminal 60 is a terminal operated by a manager K who belongs to an organization that maintains and manages the AED 1. For example, when the AED 1 has an abnormality, the management server 50 transmits information indicating the abnormality in the AED 1 to the administrator terminal 60. It should be noted that if the management server 50 is installed in a defibrillation-related maintenance center or the like, the information indicating the abnormality in the AED 1 may be shown on a display or the like connected or integrated with the management server 50.

In the defibrillation management system 100 shown in FIG. 1, one AED 1 is shown for convenience of explanation, but a plurality of AEDs 1 may be arranged around the repeater 20. In this case, information transmitted from the plurality of AEDs 1 is transmitted to the management server 50 via the repeater 20. In this manner, if a plurality of AEDs 1 are arranged around the repeater 20, it is not necessary to provide each AED 1 with a function of SIM communication such as LTE or 5G, so communication cost of each AED 1 can be reduced.

In the defibrillation management system 100 according to present embodiment, the AED 1 is communicatively connected to the management server 50 via the repeater 20. However, the present embodiment is not limited thereto. For example, the AED 1 may be communicatively connected to the management server 50 without going through the repeater 20. In this case, each AED 1 may have a wireless communication module for performing wireless communication with the wireless base station 30 by the X generation mobile communication system.

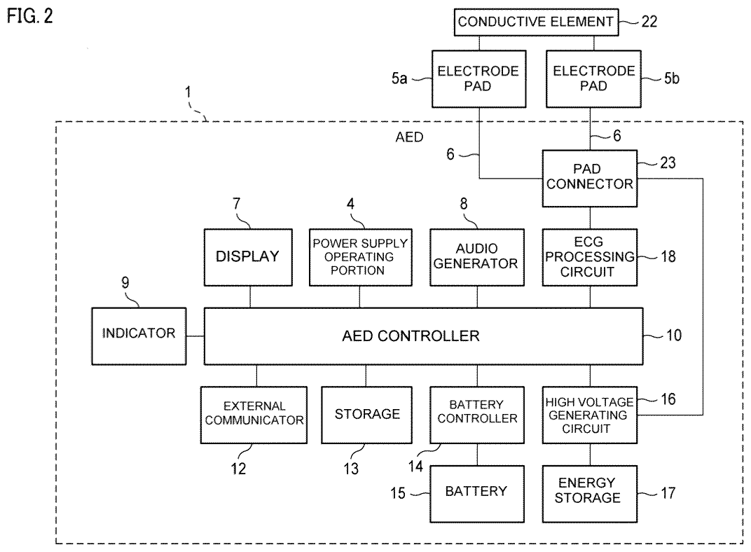

Next, the configuration of AED 1 will be described below with reference to FIG. 2. FIG. 2 is a block diagram illustrating the configuration of the AED 1. As shown in FIG. 2, the AED 1 includes an AED controller 10, a high voltage generating circuit 16, an energy storage 17, a battery 15, a battery controller 14, a storage 13, and an external communicator 12. The AED 1 further includes an ECG processing circuit 18, a pad connector 23, a sound generator 8, a power supply operating portion 4, a display 7 and an indicator 9.

The AED 1 is configured to measure an electrocardiogram of a patient (subject) who has undergone cardiac arrest due to ventricular fibrillation, and to perform defibrillation processing of applying the electric shock for the defibrillation to the heart of the subject.

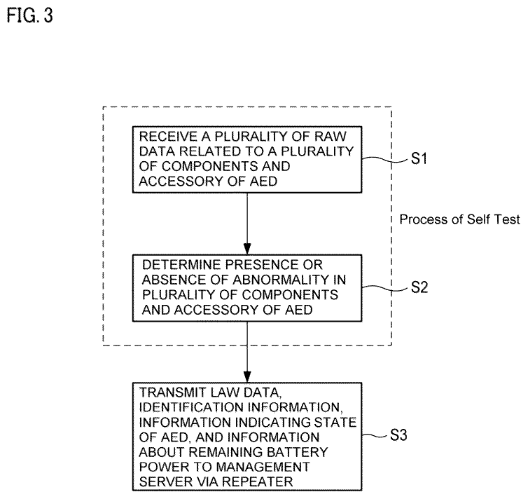

The AED controller 10 is configured to control the respective components provided in the AED 1. The AED controller 10 is, for example, constituted by a microcontroller including a processor and a memory; and an analog electronic circuit including at least an AD convertor (analog-to-digital conversion circuit). The processor includes at least one of, for example, a CPU (Central Processing Unit), an MPU (Micro Processing Unit), and a GPU (Graphics Processing Unit). The memory includes an ROM (Read Only Memory) and an RAM (Random Access Memory). The processor may be configured to expand, onto the RAM, a program (computer-readable instruction) designated from various programs incorporated in the storage 13 or the ROM and to execute various processes (e.g. a series of processes shown in FIG. 3) in cooperation with the RAM.

The high voltage generating circuit 16 includes a charge control circuit and a discharge control circuit. The charge control circuit is configured to charge the energy storage 17 with electric energy for applying the electric shock for defibrillation to the patient (subject). The discharge control circuit is configured to discharge the electric energy stored in the energy storage 17. The energy storage 17 is configured to store electrical energy for applying the electric shock for defibrillation to the patient. For example, the energy storage 17 may be a high-voltage film capacitor including a plurality of dielectric films. The electrical energy discharged from the energy storage 17 is output from the pair of electrode pads 5a and 5b via the high voltage generating circuit 16 and the pad connector 23.

The battery 15 has a battery main body and a battery memory. The battery main body functions as the power supply configured to feed electric power to the respective components of the AED 1. For example, the battery main body is a lithium primary battery. Information relevant to the battery, such as a remaining capacity of the battery is stored in the battery memory. The battery 15 may be replaceable. In the present example, the battery 15 is defined as a constituent component of the AED 1. However, the battery 15 may be defined as a separate constituent component (accessory) from the AED 1.

The battery controller 14 is provided with a circuit (such as a switching regulator or a series regulator) configured to convert the voltage of the battery 15 into a voltage required for the respective components of the AED 1. Moreover, the battery controller 14 is configured to transmit a signal indicating the voltage of the battery 15 to the AED controller 10. The AED controller 10 may first determine usage of the battery 15 based on the signal transmitted from the battery controller 14, and then integrate the usage of the battery 15 to thereby determine the remaining capacity of the battery 15.

The storage 13 is configured to store the various programs for controlling the AED 1, image data displayed on the display portion 7, audio data outputted from the sound generator 8, ECG data of the patient, data relevant to a usage state of the AED 1 (such as the remaining capacity of the battery and presence/absence of abnormality of the AED 1), etc. The storage 13 is, for example, constituted by a flash memory or a hard disk.

The external communicator 12 is configured to transmit information related to the AED 1 to the management server 50 via the repeater 20. The external communicator 12 may be a wireless communication module supporting short-range wireless communication standards such as Bluetooth (registered trademark) and Wi-Fi (registered trademark). The wireless communication module includes a transmitting and receiving antenna, a high frequency circuit, and a signal processing circuit. The external communicator 12 is configured to transmit a raw data indicating voltage, current, and/or time which are related to a plurality of components configuring the AED 1.

The ECG processing circuit 18 is configured to process ECG signals outputted from the pair of electrode pads 5a and 5b attached to the patient. For example, the ECG processing circuit 18 may have a differential amplifier that amplifies a difference between an ECG signal outputted from one of the pair of electrode pads 5a and 5b and an ECG signal outputted from the other electrode pad, and an AD converter that converts the amplified difference between the ECG signals from an analog signal into a digital signal.

The pair of electrode pads 5a and 5b are connected to the pad connector 23 through a pad cable 6. The pair of electrode pads 5a and 5b are removably attached to the AED 1, and may be disposable. Before using the AED 1, the pair of electrode pads 5a and 5b may be received in a not-shown package. When using the AED 1, the pair of electrode pads 5a and 5b are attached to the surface of the body of the patient. While the ECG signals of the patient are inputted to the pair of electrode pads 5a and 5b, electrical energy for delivering the electric shock to the heart of the patient is outputted from the pair of electrode pads 5a and 5b. In the present example, the electrode pads 5a and 5b are defined as accessories rather than components of the AED 1, but may be defined as the components of the AED 1.

The sound generator 8 (an example of an output portion) is a speaker configured to audibly output voice guidance and a warning sound relevant to an operation of the AED 1 or treatment on the patient. The power supply operating portion 4 is configured to accept an operation for turning on or off the power supply of the AED 1 from an operator. In the present example, the power supply operating portion 4 accepts the power-on operation or the power-off operation in response to the opening or closing operation of the cover 2.

The display portion 7 (another example of the output portion) is configured to display, on a display screen, guidance relevant to the operation of the AED 1 or the treatment on the patient. The display portion 7 is, for example, constituted by a liquid crystal display, an organic EL display, or the like. The indicator 9 is configured to display the status of the AED 1. For example, the indicator 9 lights up in a first display color when the AED 1 is ready for use. On the other hand, the indicator 9 may light up in a second display color different from the first control when the AED 1 is not ready for use. In addition, an indicator indicating the remaining capacity of the battery may be provided in the AED 1.

Processes of the self-test executed by the AED I will be described below with reference to FIG. 3. FIG. 3 is a flow chart for describing the processes of the self-test executed by the AED 1. As shown in FIG. 3, in step S1, the AED controller 10 receives the raw data indicating voltage, current, and/or time which are related to the plurality of components of the AED 1 and the accessory of the AED 1.

The plurality of components of AED 1 include the battery 15, the sound generator 8, an internal electronic circuit of AED 1, and the like. The internal electronic circuit of the AED 1 includes the battery controller 14, the high voltage generating circuit 16, the energy storage 17, an ECG processing circuit 18, and the like. The accessory of AED 1 includes electrode pads 5a and 5b, a pad cable 6, and the like.

Specifically, the AED controller 10 may receive voltage data related to a voltage value of the battery 15 as raw data AD1. Further, the AED controller 10 may receive current data related to a current value of current flowing through the battery controller 14 as raw data AD2. Presence or absence of the abnormality in the battery 15 can be detected based on the raw data AD1 and AD2.

The AED controller 10 may receive voltage data indicating a voltage value at a predetermined point in an electric path passing through at least the electrode pads 5a and 5b, a conductive element 22 and the pad connector 23 as raw data AD3. Presence or absence of the abnormality in the pad cable 6 or the electrode pads 5a and 5b can be detected based on the raw data AD3. The AED controller 10 may receive voltage data indicating a voltage value at a predetermined point in an electrical path between the audio generator 8 and the AED controller 10 as raw data AD4. Presence or absence of the abnormality in the audio generator 8 can be detected based on the raw data AD4.

The AED controller 10 may receive time data related to time constants of an amplifier and a filter circuit configuring the ECG processing circuit 18 as raw data AD5. Presence or absence of the abnormality in the ECG processing circuit 18 can be detected based on the raw data AD5. The AED controller 10 may receive time data indicating a time required to fully charge the energy storage 17 with electric energy as raw data AD6. Furthermore, the AED controller 10 may receive time data indicating a time required to completely discharge the electric energy from the energy storage 17 as raw data AD7. Presence or absence of the abnormality in the energy storage 17 and the high voltage generating circuit 16 can be detected based on the raw data AD6 and AD7.

Since an AD converter is supplied with the AED controller 10, the AED controller 10 can receive the raw data AD1 to AD7 as digital data. The type of raw data related to the components received by the AED controller 10 described above is one example. The AED controller 10 may receive raw data related to other components as digital data in order to detect presence or absence of an abnormality in other components of the AED 1 (for example, the indicator 9, the display 7, etc.). The number of raw data of the components received in one self-test is not particularly limited.

Next, in step S2, the AED controller 10 determines abnormality of a plurality of components and accessory. In this view point, the AED controller 10 may detect presence or absence of the abnormality in the plurality of components and the accessory based on comparison of the received raw data related to the components or the accessory data and a predetermined threshold. For example, the AED controller 10 may determine whether the voltage value of the battery 15 (the value of the raw data AD1) is equal to or greater than the predetermined threshold, and then determine whether the battery 15 is abnormal based on the determination result. Specifically, the AED controller 10 may determine that the battery 15 is abnormal when the received voltage data is smaller than the predetermined threshold. On the other hand, the AED controller 10 may determine that the battery 15 is normal when the received voltage data is equal to or greater than the predetermined threshold. When the AED controller 10 determines that the battery 15 is abnormal, the AED controller 10 may change the visual aspect of the indicator 9 (for example, luminescence color, etc.). In this manner, a user of the AED 1 can easily grasp the abnormality of the battery 15 by seeing change of the visual aspect of the indicator 9.

Further, the AED controller 10 may determine whether the time required to fully charge the energy storage 17 with electric energy (the value of the raw data AD6) is equal to or greater than a predetermined threshold value, and then the AED controller 10 may determine whether there is an abnormality in at least one of the energy storage 17 and the high voltage generating circuit 16 based on the determination result. Specifically, when the received time is equal to or greater than the predetermined threshold, the AED controller 10 may determine that at least one of the energy storage 17 and the high voltage generating circuit 16 is abnormal. On the other hand, when the received time is smaller than the predetermined threshold, the AED controller 10 may determine that both the energy storage 17 and the high voltage generating circuit 16 are normal.

In this manner, the AED controller 10 can determine presence or absence of the abnormality in the components or accessory based on the comparison of the value of the raw data and the corresponding threshold. When at least one of the plurality of components and accessory (for example, electrode pads, etc.) is abnormal, the AED controller 10 may determine that the AED 1 has an abnormality. Also, when all the components and accessory are normal, the AED controller 10 may determine that the AED 1 is normal.

Next, in step S3, the AED controller 10 transmits the raw data (for example, raw data AD1 to AD7) of the plurality of components and accessory, identification information of the AED 1, information indicating the state of the AED 1, and information about remaining battery power to the management server 50 via the repeater 20. These information may be transmitted from the AED 1 to the management server 50 at predetermined interval (for example, every day). In other words, the AED controller 10 may execute the series of processes shown in FIG. 3 at a predetermined interval (for example, every day). Further. the AED controller 10 may execute a series of processes shown in FIG. 3 according to the operation of a button (not shown) provided on the AED 1. Here, the identification information (ID information) of AED1 is information for identifying AED1. The information indicating the state of the AED 1 is information indicating presence or absence of the abnormality in the AED 1. For example, when the AED controller 10 determines that the AED 1 is abnormal as a determination result in step S2, information indicating the abnormality of the AED 1 is transmitted to the management server 50. On the other hand, when the AED controller 10 determines that the AED 1 is normal, information indicating that the AED 1 is normal is transmitted to the management server 50. The information about remaining battery power is information indicating the remaining amount of the battery 15. The AED controller 10 receives the information about remaining battery power stored in the battery memory of the battery 15 and then transmits the information about remaining battery power to the management server 50. The information about remaining battery power is indicated, for example, within a range from 0% to 100%.

After receiving the information from the AED 1, the management server 50 stores the information as table data in the storage. FIG. 4 shows an example of the defibrillation management table data 120 stored in the storage of the management server 50. It should be noted that various information included in the defibrillation management table data 120 is merely an example.

The management server 50 includes at least a processor, a memory, a storage, and a network communication unit.

The management server 50 may send an e-mail including the AED identification number and the information indicating the abnormality in the AED 1 to the mail address of the manager K when the management server 50 receives the information indicating the abnormality of the AED 1. In this manner, the administrator terminal 60 shown in FIG. 1 can receive information indicating an abnormality of the AED 1 from the management server 50 via the receiving mail server (not shown). Further, when the management server 50 receives information indicating that the remaining battery level is less than a predetermined level, the management server 50 may send an e-mail including the AED identification number and information indicating that the remaining battery level is low to the mail address of the manager K. In this manner, the administrator terminal 60 can receive information indicating that the remaining battery level is low from the management server 50 via the receiving mail server.

In addition, the management server 50 can automatically perform applied data analysis such as abnormality prediction of the AED 1 based on the raw data AD1 to AD7. For example, the management server 50 may predict the abnormality prediction in the AED1 based on the raw data AD1 to AD7 using a machine learning model which defines the raw data AD1 to AD7 as an input layer, and information related to the abnormality prediction of the AED1 (for example, probability of abnormality prediction in the AED1) as an output layer. Further, the management server 50 may predict the abnormality in the AED1 based on the raw data AD1 to AD7 using a predetermined abnormality prediction algorithm. When probability of the abnormality in the AED 1 is high as a result of the abnormality prediction analysis, the management server 50 may send an e-mail including the AED identification number and information related to the abnormality prediction of the AED 1 to the email address of the manager K.

According to this embodiment, the raw data AD1 to AD7 related the plurality of components configuring the AED 1 and the accessory of the AED 1 are transmitted to the management server 50 via the repeater 20. In this manner, data analysis such as the abnormality prediction in the AED 1 can be performed by effectively using the raw data AD1 to AD7 transmitted from the AED 1 in the management server 50. In this view point, in a conventional remote maintenance system, applied data analysis such as abnormality prediction in the AED 1 cannot be performed, although the management server 50 can detect the abnormality in the AED 1 based on the information indicating presence or absence of the abnormality in the AED 1. On the other hand, in the AED 1 according to this embodiment, since it is possible to transmit the raw data AD1 to AD7 related to at the plurality of components and the accessory to the management server 50, the management server 50 can perform the data analysis such as abnormality prediction in the AED 1 by effectively using the raw data AD1 to AD7 transmitted from the AED 1. In this manner, it is possible to provide the AED 1 and the defibrillation management system 100 that can effectively use the raw data generated from components, etc. (especially the battery 15, the electrode pads 5a and 5b, and internal electronic circuit) of the AED 1.

Further, after the management server 50 predicts the abnormality of the AED 1 based on the raw data AD1 to AD7, information related to the abnormality prediction of the AED 1 is transmitted to the administrator terminal 60 via the communication network 40. In this manner, the manager K who maintains and manages the AED 1 can grasp the information related to the abnormality prediction of the AED 1 through the administrator terminal 60, so it is possible to inspect the AED 1 before AED 1 breaks down. In this manner, it is possible to prevent a case in which the AED 1 cannot be used due to the abnormality when the AED 1 is actually used.

Further, in this embodiment, the AED 1 instead of the management server 50 determines the presence or absence of abnormality in the plurality of components based on the raw data of the plurality of components. Therefore, even if a communication failure occurs between the AED 1 and the management server 50, it is possible to surely determine presence or absence of the abnormality in the AED 1. In addition, compared to the case where presence or absence of the abnormality in the AED 1 is determined in the management server 50, it is possible to surely and rapidly determine presence or absence of the abnormality in the AED 1 without depending on communication status between the AED 1 and the management server 50.

The embodiment of the present disclosure has been described above. However, the technical scope of the present disclosure should not be limitedly interpreted by the description of the present embodiment. The present embodiment is merely exemplified, and it is understood by those skilled in the art that various modifications can be made on the embodiment within the scope of the invention described in CLAIMS. The technical scope of the present disclosure should be defined based on the scope of the invention described in the CLAIMS and its equivalent scopes.

In this embodiment, the AED controller 10 determines presence or absence of the abnormality in the plurality of components of the AED 1 based on the raw data of the plurality of components. The management server 50 may determine presence or absence of the abnormality instead of the AED controller 10. In this case, the AED 1 may not transmit the information indicating the state of the AED 1 to the management server 50. The management server 50 may determine presence or absence of the abnormality in the AED 1 upon determining presence or absence of the abnormality in each component based on the raw data transmitted from the AED 1. In addition, in this embodiment, an AED 1 is described as an example of the defibrillator of the present invention. However, the defibrillator is not limited to the AED. For example, a defibrillator (such as a semi-automatic defibrillator) installed in a hospital may be applied as the defibrillator of the present invention. In this case as well, the raw data of the components of the defibrillator are transmitted to the management server 50.

This application is based on Japanese Patent Application No. 2022-011702 filed on Jan. 28, 2022, the entire contents of which are incorporated herein by reference.

1. A defibrillator configured to execute a defibrillation process of delivering an electric shock for defibrillation to a heart of a subject, the defibrillator comprising:
a processor; and
a memory that stores a computer-readable instruction that when executed by the processor, causes the defibrillator to perform operations comprising:
receiving a raw data related to at least one of a plurality of components configuring the defibrillator and an accessory of the defibrillator; and
transmitting the received raw data and an identification information of the defibrillator to a management server,
wherein the raw data is used to determine presence or absence of an abnormality in at least one of the plurality of components and the accessory.

2. The defibrillator according to claim 1,
wherein the defibrillator determines presence or absence of the abnormality in at least one of the plurality of components and the accessory based on the received raw data, and
wherein the defibrillator transmits an information indicating presence or absence of the abnormality in the defibrillator with the raw data and the identification information to the management server.

3. The defibrillator according to claim 2,
wherein the defibrillator determines presence or absence of the abnormality in at least one of the plurality of components and the accessory based on comparison of the raw data and a threshold related to the raw data.

4. The defibrillator according to claim 1,
wherein the defibrillator receives a plurality of raw data each related to a corresponding one of the plurality of components and the accessory;
wherein the defibrillator transmits the plurality of raw data and the identification information of the defibrillator to the management server;
wherein the defibrillator determines presence or absence of the abnormality in the plurality of components and the accessory based on the plurality of raw data; and
wherein the defibrillator transmits an information indicating the abnormality in the defibrillator to the management server, when at least one of the plurality of components and the accessory is abnormal.

5. The defibrillator according to claim 1,
wherein the plurality of components includes a battery configured to supply electric power to the defibrillator and an internal electronic circuit of the defibrillator; and
wherein the accessory includes an electrode pad to which an electrocardiogram signal of the subject is input and from which electric energy for applying the electric shock is output.

6. The defibrillator according to claim 1,
wherein at least a part of the raw data indicates voltage, current, and/or time which are related to at least one of the plurality of components and the accessory.

7. The defibrillator according to claim 1,
wherein the plurality of components includes an energy storage configured to store electric energy for delivering the electric shock; and
wherein at least a part of the raw data indicates a time required to fully charge the energy storage with the electric energy.

8. A defibrillator management system comprising:
the defibrillator according to claim 1; and
the management server communicatively connected to the defibrillator.

9. The defibrillator management system according to claim 8, further comprising a repeater communicatively connected to the defibrillator by short-range wireless communication,
wherein the repeater is communicatively connected to the management server.
